(19) United States
(12) Patent Application Publication
(10) Pub. No.: US 2004/0235942 A1
(43) Pub. Date: Nov. 25, 2004

(54) Method for preventing recurring venous thromboembolism using long-term low-intensity warfarin

(76) Inventors: Paul M. Ridker, Chestnut Hill, MA (US); Samuel Z. Goldhaber, Chestnut Hill, MA (US); Robert J. Glynn, Belmont, MA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/767,758

(22) Filed: Jan. 29, 2004

Related U.S. Application Data
(60) Provisional application No. 60/443,828, filed on Jan. 30, 2003.

(51) Int. Cl. A61K 31/366

(52) U.S. Cl. 514/457

(57) ABSTRACT

A method is provided for inhibiting, preventing or reducing the incidence of venous thromboembolism (VTE) in a patient who previously has undergone standard therapy for VTE, that is, a dose of warfarin of 2 to 3 INR over a 3 to 12 month period, wherein the patient is administered a low dose of warfarin of less than 2 INR and preferably from about 1.4 to less than 2 INR over an extended period of time.

This application claims priority from U.S. Provisional Application 60/443,828 filed Jan. 30, 2003 which is incorporated herein by reference.

### FIELD OF THE INVENTION

The present invention relates to a method for inhibiting or preventing recurrent venous thromboembolism (VTE) using low-intensity warfarin.

### BACKGROUND OF THE INVENTION

Therapy for idiopathic venous thromboembolism (VTE) typically includes a 5 to 10 day course of intravenous or subcutaneous heparin followed by a 3 to 12 month period of oral anticoagulation with full dose warfarin, adjusting the dosage to an International Normalized Ratio (INR) between 2.0 and 3.0 (1-4). After cessation of anticoagulation, however, recurrent VTE is a major clinical problem with rates estimated between 6 and 9 percent annually (5,6). Unfortunately, no therapy with an acceptable benefit to risk ratio is available to provide VTE patients with a long-term management strategy. In particular, while extended use of full-dose warfarin is associated with reduced rates of recurrent deep vein thrombosis and pulmonary embolism (2-4), community based studies have consistently found this approach to be associated with substantial risk of major hemorrhage. For example, in observational studies, use of full-dose warfarin is associated with major bleeding rates between 5 and 9 percent (7-9). Similarly, an annual major hemorrhage rate of 3.8 percent was observed in a recent full-dose warfarin trial despite careful on-site anticoagulation monitoring (3).

By contrast, low-intensity warfarin has been shown in several settings to have a low risk of bleeding when used on a chronic basis and may require less frequent monitoring. Further, experimental data indicate that low-intensity warfarin is effective in reducing biochemical markers of coagulation such as factor VII activity and levels of prothrombin fragment F₁₊₂ (10,11). No clinical data, however, are available evaluating low-intensity warfarin for long-term venous thrombosis prophylaxis, although this approach has been used successfully for the prevention of first thrombosis among patients with indwelling central venous catheters and among women with metastatic breast cancer (12,13).

### DESCRIPTION OF THE INVENTION

It has now been found that use of low-intensity warfarin over a long term significantly reduces incidence of recurrent venous thromboembolism (VTE).

In accordance with the present invention, a method is provided for inhibiting, preventing or reducing incidence of recurrent venous thromboembolism (VTE) in a patient who has previously undergone standard therapy for VTE involving 3 to 12 months of full-dose warfarin using a targeted International Normalized Ratio [INR] between 2.0 and 3.0. The method of the invention includes the step of administering to a patient who has previously undergone standard therapy for VTE a low dose of warfarin which is lower than the standard dose of warfarin administered in standard treatment of VTE. Thus, the low dose of warfarin will be less than 2 INR and will be within the range from 1.4 to less than 2.0 INR and is preferably from about 1.6 to about 1.8 INR, and most preferably about 1.7 INR, and will comprise from about 0.5 to about 10 mg daily and preferably from about 3 to about 6 mg daily, and most preferably about 4 mg daily. The low-dose warfarin will be continued over an extended period of time of from at least 2 to 5 years up to the life of the patient.

### BRIEF DESCRIPTION OF THE FIGURES

FIG. 1 is a graph showing distribution of INR levels at the 2 month follow up visits, according to randomized treatment assignment.

FIG. 2 is a graph showing cumulative risk of recurrent VTE (left); and

FIG. 3 is a graph showing composite study endpoint of recurrent VTE, major hemorrhage, and all-cause mortality (right).

### EXAMPLE

### Summary of Trial

### Background

Standard therapy for venous thromboembolism (VTE) includes 3 to 12 months of full-dose warfarin using a targeted International Normalized Ratio [INR] between 2.0 and 3.0. However, while recurrent VTE is common following cessation of this regimen, no therapeutic agent has shown an acceptable benefit-to-risk ratio for long-term management.

### Methods

In this double-blind trial, patients with idiopathic VTE who had received standard full-dose anticoagulation for at least 3 months were randomly assigned to placebo or to long-term, low-intensity warfarin using a targeted INR of 1.5 to 2.0. All participants were followed for recurrent VTE, major bleeding events, and all-cause mortality.

### Results

An interim review by the Independent Data and Safety Monitoring Board led to early termination of the trial after 508 patients had been randomized and followed for up to 4.3 years (mean 2.1 years). Of 253 patients assigned to placebo, 37 had recurrent VTE (7.3 per 100 person years) as compared to 14 of 255 assigned to low-intensity warfarin (2.6 per 100 person years), a risk reduction of 64 percent (hazard ratio 0.36, 95% Cl 0.20 to 0.67, P=0.0007). Risk reductions were similar in magnitude for all pre-specified subgroups, including those with and without factor V Leiden and the G20210A prothrombin polymorphism. Major bleeding complications occurred in 2 patients allocated to placebo and 5 allocated to low-intensity warfarin (P=0.25). Death occurred in 8 patients allocated to placebo and 4 allocated to low-intensity warfarin (P=0.27). 2 deaths were due to pulmonary embolism and 1 to hemorrhagic stroke, all in the placebo group. Low-intensity warfarin was thus associated with a 48 percent reduction in the pre-specified composite study endpoint of recurrent VTE, major hemorrhage, or death (hazard ratio 0.52, 95% Cl 0.31 to 0.87, P=0.011). In on-treatment analyses, the reduction in risk of recurrent VTE was between 77 and 81 percent.

### Conclusion

Low-intensity warfarin is a highly effective and safe method to prevent recurrent deep-vein thrombosis and pulmonary embolism.

### DETAILED DESCRIPTION OF TRIAL

With finding from the National Heart, Lung, and Blood Institute (NHLBI) (HL-57951 and HL-58036), the Prevention of Recurrent Venous Thromboembolism (PREVENT) trial was initiated in July 1998 to test the hypothesis that long-term, low-intensity warfarin (target INR 1.5 to 2.0) might provide a safe and effective method to reduce the risk of recurrent VTE among individuals who had suffered a prior idiopathic VTE (14). As a secondary aim, the study was also designed as a pharmacogenetic trial testing the hypothesis that individuals with thrombophilic mutations such as factor V Leiden or the G20210A prothrombin polymorphism might differentially benefit from long-term, low-intensity warfarin prophylaxis.

The PREVENT trial was designed to enroll 1750 patients for an average follow-up period of 4 years. As described here, the trial was terminated by its Independent Data and Safety Monitoring Board after 508 patients were randomized due to the emergence of a statistically extreme benefit of low-intensity warfarin in the absence of any substantial evidence of harm.

### Methods

Patients

Men and women age 30 and over with documented idiopathic venous thromboembolism were eligible if within the past two years they had completed at least three uninterrupted months of oral anticoagulation with full-dose warfarin. All index events were confirmed by objective criteria at the central Clinical Coordinating Center based upon venography, compression ultrasound or MRI reports (for deep vein thrombosis) or by ventilation/perfusion scans, angiograms, or chest computed tomography scans (for pulmonary embolism). Idiopathic events were defined as those not occurring within 90 days of surgery, trauma, or a diagnosis of metastatic cancer. Patients were ineligible for the trial if they had a history of metastatic cancer, major gastrointestinal bleeding, hemorrhagic stroke, or life expectancy of less than 3 years. Participants with a daily requirement for >325 mg aspirin, dipyridamole, ticlopidine, clopidogrel, heparin, drugs which affect the prothrombin time, or who had a known lupus anticoagulant or antiphospholipid antibodies were excluded. Every patient gave written informed consent and the study protocol was approved by each institutional review board.

Open Label Run in, INR Evaluation, Randomization and Dose Adjustment

Eligible patients were entered into a 28 day open-label run-in period designed to ensure that all participants could be titrated to a stable warfarin dose that achieved an INR between 1.5 and 2.0 prior to randomization without exceeding 10 mg/day. The run-in phase was also used to exclude individuals with less than 80 percent compliance.

During the run-in, at randomization, and throughout follow-up, all INR assessments at each study site were made using specially designed fingerstick devices with an identical thromboplastin [International Sensitivity Index 2.0] (CoaguChek, Roche Diagnostics, Indianapolis, Ind.). These devices were altered electronically to provide a coded study value which was transmitted to the Data Coordinating Center. All dose adjustments were made in a blinded manner using a simple clinical algorithm (Appendix A). Whenever a dose-adjustment greater than 1 mg of warfarin daily was required or whenever randomized therapy was temporarily stopped, participants returned for repeat blinded INR evaluation within seven days.

Randomization to low-intensity warfarin (Coumadin, Bristol-Myers Squibb, targeted INR 1.5 to 2.0) or to matching placebo was performed centrally. Randomization was stratified by clinical site, time since index event (≦6 months, >6 months) and by whether the index event was the patient's first ever VTE. All participants were then followed with office visits once every two months that included blinded INR evaluations and dose adjustments. To further ensure blinding, sham dose adjustments were made in the placebo group.

Follow-Up and Study Endpoints

As PREVENT was specifically designed to evaluate clinically relevant recurrent thromboembolic events, no surveillance for asymptomatic thrombosis was undertaken. Rather, at each visit the occurrence of any incident clinical events since the last visit two months earlier was evaluated. All endpoints were reviewed by a committee of physicians unaware of study drug assignment. The endpoint of recurrent deep vein thrombosis was confirmed if there was a positive venography study, doppler compression ultrasound, or MRI. Events documented by clinical diagnosis alone were not confirmed. The endpoint of pulmonary embolism was confirmed if there was a positive angiogram, a ventilation-perfusion scan which showed at least two segmental defects without ventilation defects, or clear evidence of thrombosis documented by chest CT or MRI. In cases where the recurrent DVT or PE was ipsilateral to the index event, clear documentation demonstrating interval change was required. Major hemorrhage was defined as any bleeding episode that led either to hospitalization or transfusion.

As an index of net clinical benefit, we defined on an a priori basis a composite endpoint of recurrent VTE, major hemorrhage, and all-cause mortality. Incident stroke events were also monitored. These were classified as hemorrhagic or thromboembolic on the basis of clinical records or CT/MRI scanning which were available for all episodes. To avoid double-counting, hemorrhagic strokes were considered as major hemorrhages in analyses using the combined endpoint of recurrent thrombosis, major hemorrhage, and all-cause mortality.

Genetic Analyses

Blood samples obtained at enrollment underwent DNA extraction and were evaluated for factor V Leiden and the G20210A prothrombin polymorphism in a central laboratory where personnel were unaware of randomized treatment assignment. Genetic data were not made available to the clinical sites or to members of the endpoints committee.

Trial Monitoring and Statistical Analysis

The NHLBI appointed an Independent Data and Safety Monitoring Committee (IDSMC). The IDSMC chose to monitor the trial with a Lan-DeMets procedure with O'Brien and Fleming spending function (15). At the pre-specified review of the trial upon accrual of approximately 40 percent of the expected information in the planned study, the IDSMC voted on Dec. 4, 2002 to stop the trial based on strong evidence of efficacy and crossing of the monitoring boundary. The Director of the NHLBI accepted this recommendation.

Comparisons between the warfarin and placebo groups in the distributions of continuous variables used Wilcoxon rank sum tests; for categorical variables, comparisons used Chi-square tests. The primary analysis was an intention-to-treat comparison of the difference between time to first confirmed recurrent VTE after randomization in the two treatment arms using a two-sided log-rank test. The method of Kaplan and Meier was used to estimate the probability of recurrence by time within treatment groups. Estimation of the number of patients needed to treat (NNT) to prevent one recurrent event was based on rates at one year. We used the proportional hazards model for estimation of the relative hazard of recurrent VTE associated with low-intensity warfarin treatment and obtained confidence intervals from this model. The hypothesis of a varying effect of treatment over time was tested in a proportional hazards model including an interaction of treatment with time. Tests and estimates of treatment effects on the composite endpoint used the same methods.

The primary pre-specified subgroup analysis evaluated the effect of treatment separately in persons with and without either factor V Leiden or the prothrombin mutation. The hypothesis of a different effect of treatment by genetic status was tested in a proportional hazards model including the interaction of treatment with the presence of either factor V Leiden or G20210A prothrombin mutation. Other descriptive comparisons within subgroups used the same methods.

### Results

Patients Therapy, and INR Evaluations

Between Jul. 6, 1998 and Dec. 4, 2002, 571 patients entered the 28 day run-in period. At the time of early termination, 508 patients had completed the 28 day run-in and been randomized into the trial, 253 allocated to placebo and 255 allocated to low-intensity warfarin. Median duration of full-dose anticoagulation completed prior to study enrollment was 6.5 months.

Clinical characteristics and known risk factors for recurrent VTE were equally distributed between the low-intensity warfarin and placebo groups (Table 1). 47 percent of the study cohort were women. Median age of participants was 53 years. 28 percent carried either factor V Leiden or the prothrombin mutation. Of note, median body mass index was high (29.9 kg/m²).

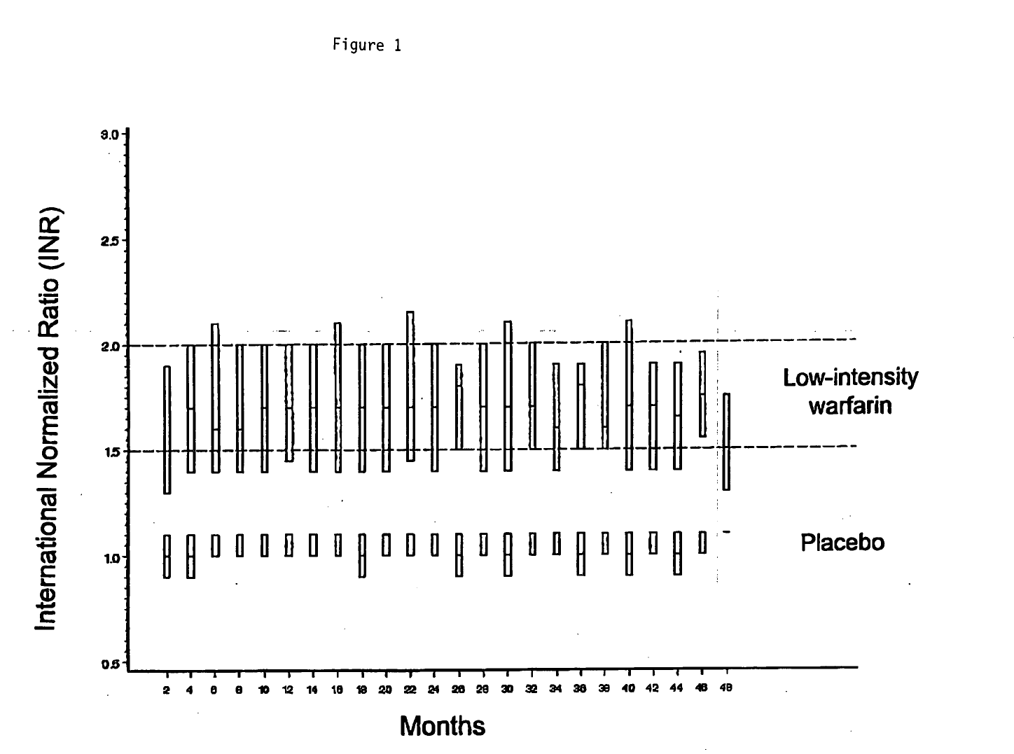

Mean duration of follow up after randomization was 2.1 years, with a maximum treatment duration of 4.3 years. The median INR of patients in the placebo group was 1.0 (interquartile range 1.0 to 1.1) while the median INR in the low-intensity warfarin group was 1.7 (interquartile range 1.4 to 2.0). In the active therapy group, median warfarin dose was 4 mg (interquartile range 3 to 6 mg), with an absolute range between 0.5 and 10.0 mg daily. As shown in FIG. 1 of this difference in INR levels between the placebo and active therapy groups was maintained throughout the study period. The percentages of patients attending scheduled follow-up visits never differed between treatment groups at any point in the trial.

Recurrent Venous Thromboembolism

In total, there were 51 confirmed events of recurrent VTE after randomization. Of these, 39 were deep vein thrombosis only and 12 were associated with pulmonary embolism. Eighty-six percent of all recurrent VTE were idiopathic while 14 percent were associated with a new diagnosis of cancer, recent surgery, or trauma.

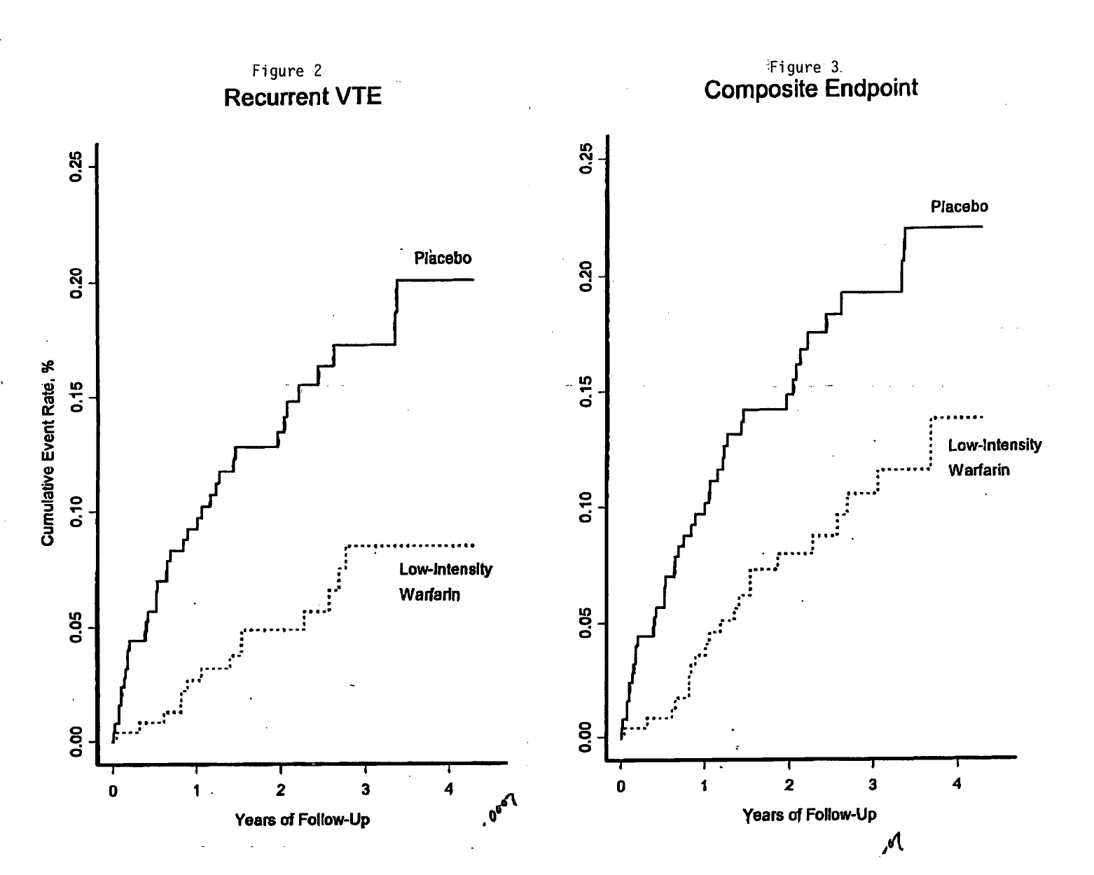

Of the 253 patients allocated to placebo, 37 had a confirmed recurrent VTE (7.3 per 100 person years) as compared to 14 of 255 assigned to low-intensity warfarin (2.6 per 100 person years), a risk reduction of 64 percent (hazard ratio 0.36, 95% Cl 0.20 to 0.67, P=0.0007) (Table 2). The cumulative probability of recurrent VTE is shown in FIG. 2 (left). Low-intensity warfarin had similar efficacy in the prevention of early as well as late recurrent events. Based on these rates, 14 patients need to be treated for one year to prevent one recurrent event.

Of 77 individuals affected by either factor V Leiden or the prothrombin mutation who were assigned placebo, 14 had recurrent VTE (8.6 events per 100 person years), as compared to 3 of 66 assigned to low-intensity warfarin (2.2 events per 100 person years) (Table 3). This 75 percent relative risk reduction among those with inherited thrombophilias (hazard ratio=0.25, 95%Cl 0.07 to 0.87) was not significantly different from the 58 percent risk reduction among those without factor V Leiden or the prothrombin mutation (hazard ratio 0.42, 95% Cl 0.20 to 0.86) (P for interaction=0.50).

Risk reductions were of similar magnitude in other subgroups evaluated (Table 3). Among women, low-intensity warfarin was associated with a 80 percent reduction in risk of recurrent VTE (hazard ratio=0.20, 95% Cl 0.06 to 0.68) whereas a 53 percent reduction was observed among men (hazard ratio=0.47, 95% Cl 0.23 to 0.97) (P for interaction=0.23). We observed no significant interactions between the magnitude of risk reduction and categories of age (P for interaction=0.90) or time since randomization (P for interaction=0.16).

Bleeding Episodes

There were 2 bleeding episodes requiring hospitalization among those allocated to placebo (0.4 per 100 person-years) and 5 among those allocated to low-intensity warfarin (0.9 per 100 person years), a non-significant difference (P=0.25). Of major bleeds occurring among those allocated to low-intensity warfarin, 3 were gastrointestinal, 1 was a lower-extremity hematoma, and 1 was hematuria in the setting of renal calculus removal. Only 1 major hemorrhage required transfusion of packed red blood cells; this occurred in a patient allocated to low-intensity warfarin who was receiving no protocol full-dose warfarin at the time of the bleed.

Any minor bleeding or bruising was reported among 34 patients allocated to placebo and among 60 patients allocated to low-intensity warfarin (hazard ratio 1.93, 95% Cl 1.27 to 2.94).

All Cause Mortality, Stroke, and Other Endpoints

There were 8 deaths in the placebo group and 4 in the low-intensity warfarin group (P=0.27). Two deaths were due to fatal pulmonary embolism, and 1 death was due to fatal hemorrhagic stroke, all in the placebo-allocated group. There were 2 confirmed strokes in the placebo group and 1 in the low-intensity warfarin group. As noted above, 1 stroke was hemorrhagic and occurred in a patient assigned to placebo. This participant initially was hospitalized for a thromboembolic stroke that became hemorrhagic after initiation of heparin and clopidogrel therapy.

There were 13 diagnoses of cancer during follow-up, 9 in the placebo group and 4 in the low-intensity warfarin group (P=0. 18).

Five individuals suffered myocardial infarction during follow-up, 2 in the placebo group and 3 in the low intensity warfarin group (P=0.62).

On an a priori basis, a composite study endpoint intended to reflect net clinical benefit was defined as recurrent VTE, major hemorrhage (including hemorrhagic stroke), and all-cause mortality. This composite endpoint was reduced 48 percent in the low-intensity warfarin group (hazard ratio 0.52, 95% Cl 0.31 to 0.87, P=0.011) (FIG. 3 right).

On Treatment Analyses

Study drug was discontinued before completion of follow-up in 56 patients on placebo and 62 patients on low-intensity warfarin (P=0.82). Primary reasons for study drug discontinuation were patient refusal, minor bruising, the development of other medical conditions, or a new indication for anticoagulation. Drug discontinuation for each of these reason, including minor bleeding, occurred with equal frequency in the placebo and active therapy groups.

Fifteen participants had a recurrent VTE after cessation of study drug. Of these, 8 were in the placebo group and 7 were in the low-dose warfarin group. Thus, among participants documented as being on study drug at the time of the recurrent event, the relative risk reduction associated with low-intensity warfarin was 77 percent (hazard ratio 0.23, 95% Cl 0.10 to 0.53).

No individuals with recurrent VTE who had stopped study drug were taking another form of anticoagulation at the time of the recurrent event. Thus, in an analysis based upon use or non-use of long-term anticoagulation at the time of the recurrent event, an 81 percent reduction in risk was observed in the low-intensity warfarin group (hazard ratio 0.19, 95% Cl 0.084 to 0.42).

### Discussion

This randomized, double-blind, placebo-controlled trial demonstrates that long-term, low-intensity warfarin given with a target INR between 1.5 and 2.0 results in a large and highly significant reduction in the risk of recurrent venous thrombosis: This benefit was present in all subgroups evaluated, was consistent for early as well as late recurrences, and was equally present among those with and without factor V Leiden and the G20210A prothrombin polymorphism. This reduction in risk was achieved with little evidence of any increase in risk of major hemorrhage or stroke, despite using infrequent anticoagulation monitoring. As such, findings from the PREVENT trial can be readily implemented in clinical practice, and strongly suggest that long-term use of low-intensity warfarin should be considered as a new standard of care for the management of venous thrombosis following cessation of full-dose warfarin therapy.

We believe it important to put the current data for low-intensity warfarin in perspective with data for other therapeutic approaches being considered for use in the long-term management of VTE. Previous work demonstrates that short-term use of full-dose warfarin is highly effective therapy after a first episode of VTE, and on the basis of randomized trial evidence, usual care typically includes full-dose warfarin therapy for up to 12 months (1,4). Two completed trials showed that extended use of full-dose warfarin for longer than one year continues to provide excellent efficacy in preventing recurrent VTE when compared to placebo (2,3). However, in both of these trials, major bleeding rates were high during the administration of long-term, full-dose warfarin, an observation consistent with community based concerns regarding the safety of long-term warfarin when used with an INR targeted between 2.0 and 3.0 (7-9).

Another alternative approach to full-dose warfarin for long term management is novel anticoagulants such as oral direct thrombin inhibitors. One such agent, ximelagatran, has recently been compared to placebo in a trial similar in design to PREVENT and was also highly effective (16). Ximelagatran has a relatively short half-life requiring twice daily dosing. Although this agent may not require anticoagulation monitoring, its use has been associated with elevations in hepatic transaminase levels and will require ongoing surveillance of liver function. Given the results presented here, a direct comparison of ximelagatran to low-intensity warfarin will be needed to determine if either of these therapies is superior to the other for long term management. This issue has public health consequences as the cost of ximelagatran is likely to be high in comparison to warfarin used in low-intensity regimens.

From a pathophysiologic perspective, it is important to recognize that PREVENT was specifically designed as a pharmacogenetic trial in which we pre-specified that secondary analyses would be performed among individuals with and without inherited thrombophilias such as the factor V Leiden and G20210A prothrombin polymorphisms, each of which is known to increase risk of first venous thrombosis (17-22). Whether these genetic disorders are associated with increased risk of recurrent VTE remains controversial (23-28). In PREVENT, individuals with factor V Leiden or the G20210A prothrombin polymorphism were not at substantially increased risk of recurrent VTE when compared to individuals without these disorders. Moreover the relative benefit of low-intensity warfarin in preventing recurrent events was not altered by genetic status. Thus, in this randomized intervention trial, we found no evidence of a substantial pharmacogenetic interaction. Indeed, it is uncertain in these data that screening for either factor V Leiden or the prothrombin polymorphism had any important clinical consequences either in terms of prognosis or in terms of differential therapeutic response. As the PREVENT trial excluded patients with known anti-phospholipid antibody syndrome, the efficacy of low-intensity warfarin among such individuals remains uncertain.

In sum, as demonstrated in this randomized trial, low-intensity warfarin is a highly effective method to prevent recurrent VTE. These data reinforce the importance of investigating agents that may well have clinical efficacy, yet due to their generic status provide little financial incentive for pharmaceutical industry investigation.

TABLE 1
Baseline characteristics of study participants

| | | Low-intensity | |
|---|---|---|---|
| | Placebo | Warfarin | |
| | (N = 253) | (N − 255) | P |
| Age, years* | 53 (47, 64) | 53 (46, 65) | 0.82 |
| Female (%) | 47.4 | 47.1 | 0.93 |
| Ethnicity (%) | | | |
| Caucasian | 86.6 | 88.2 | 0.32 |
| Black | 10.3 | 9.0 | |
| Hispanic | 0.8 | 2.0 | |
| Other | 2.4 | 0.8 | |
| Body Mass Index (kg/m²)* | 29.9 (26.6, 34.4) | 29.9 (26.6, 34.2) | 0.90 |
| History of Diabetes (%) | 8.7 | 6.7 | 0.39 |
| Family History of VTE (%) | 31.6 | 26.3 | 0.18 |
| Factor V Leiden (%) | 26.6 | 22.0 | 0.23 |
| Prothrombin mutation (%) | 4.8 | 4.7 | 0.98 |
| Months of full-dose | 6.4 (5.7, 9.0) | 6.7 (5.9, 10.8) | 0.15 |
| warfarin prior to | | | |
| enrollment* | | | |

*values are median (interquartile range)

TABLE 2
Major Study Endpoints According to Treatment Group

| | | | Low-Intensity | | | |
|---|---|---|---|---|---|---|
| | Placebo | | Warfarin | | Hazard Ratio | |
| Outcome | N | Rate* | N | Rate* | (95% Cl) | P |
| Recurrent VTE | 37 | 7.3 | 14 | 2.6 | 0.36 (0.20, 0.67) | 0.0007 |
| Bleeding | | | | | | |
| Major | 2 | 0.4 | 5 | 0.9 | 2.54 (0.49, 13.13) | 0.25 |
| Minor** | 34 | 6.7 | 60 | 12.9 | 1.93 (1.27, 2.94) | 0.002 |
| Deaths | 8 | 1.4 | 4 | 0.7 | 0.51 (0.15, 1.70) | 0.27 |
| Cancer | 9 | 1.6 | 4 | 0.7 | 0.46 (0.14, 1.48) | 0.18 |
| Myocardial | 2 | 0.4 | 3 | 0.5 | 1.56 (0.26, 9.32) | 0.62 |
| Infarction | | | | | | |
| Composite | 41 | 8.0 | 22 | 4.1 | 0.52 (0.31, 0.87) | 0.011 |
| Endpoint** | | | | | | |
| *Rates are given as events per 100 person years. | | | | | | |
| **Major bleeding was defined as those resulting in hospitalization, transfusion of packed red blood cells, or hemorrhagic stroke. | | | | | | |
| # The pre-specified composite endpoint includes first VTE, major bleed, or death. | | | | | | |

TABLE 3
Rates and hazard ratios for recurrent VTE in clinically important
subgroups, according to randomized treatment assignment

| | | | Low- | | | |
|---|---|---|---|---|---|---|
| | | | Intensity | | | |
| | Placebo | | Warfarin | | Hazard Ratio | Interaction |
| Characteristic | N | Rate* | N | Rate* | (95% Cl) | P-value** |
| Factor V Leiden | | | | | | |
| or Prothrombin | | | | | | |
| Mutation | | | | | | |
| Present | 14 | 8.6 | 3 | 2.2 | 0.25 (0.07, 0.87) | 0.50 |
| Absent | 23 | 6.6 | 11 | 2.7 | 0.42 (0.20, 0.86) | |
| Gender | | | | | | |
| Men | 22 | 8.6 | 11 | 4.0 | 0.47 (0.23, 0.97) | 0.23 |
| Women | 15 | 5.9 | 3 | 1.1 | 0.20 (0.06, 0.68) | |
| Age, years | | | | | | |
| 30-44 | 8 | 7.6 | 4 | 3.3 | 0.46 (0.14, 1.52) | 0.90 |
| 45-64 | 20 | 7.3 | 5 | 1.7 | 0.25 (0.09, 0.66) | |
| 65-89 | 9 | 6.9 | 5 | 3.9 | 0.55 (0.18, 1.64) | |
| Time after | | | | | | |
| randomization | | | | | | |
| ≦1 year | 22 | 10.1 | 6 | 2.7 | 0.27 (0.11, 0.66) | 0.16 |
| >1 year | 15 | 5.1 | 8 | 2.5 | 0.49 (0.21, 1.16) | |
| *Rates are given as events per 100 person years. | | | | | | |
| **The null hypothesis is that there are no differences across subgroups; for interval-scaled variables such as age and time after randomization, the interaction tested is | | | | | | |
| # between this continuous variable and treatment. | | | | | | |

### REFERENCES

1. Shulman, S., Rhedin, A-S., Lindmarker, P. et al. A comparison of six weeks with six months of oral anticoagulant therapy after a first episode of venous thromboembolism. N. Engl. J. Med. 1995; 332:1661-5.

2. Shulman, S., Granqvist, S., Holmström, M. et al. The duration of oral anticoagulant therapy after a second episode of venous thromboembolism. N. Engl. J. Med. 1997; 336-393-8.

3. Kearon, C., Gent, M., Hirsh, J. et al. A comparison of three months of anticoagulation with extended anticoagulation for a first episode of idiopathic venous thromboembolism. N. Engl. J. Med. 1999; 340:901-7.

4. Agnelli, G., Prandoni, P., Santamaria, M. G. et al. for the Warfarin Optimal Duration Italian Trial Investigators. Three months versus one year of oral anticoagulant therapy for idiopathic deep venous thrombosis. N. Engl. J. Med. 2001; 345:165.9.

5. Prandoni, P., Lensing, A. W., Cogo, A. et al. The long term clinical course of acute deep venous thrombosis. Ann. Intern. M. 1996; 125:1-7.

6. Hirsh, J. The optimal duration of anticoagulation therapy for venous thromboembolism. N. Engl. J. Med. 1995; 332:1710-1.

7. Fihn, S. D., Callahan, C. M., Martin, D. C., McDonell, M. B., Henikoff, J. G., White, R. H. for the National Consortium of Anticoagulation Clinics. The risk for and severity of bleeding complications in elderly patients treated with warfarin. Ann. Intern. Med. 1996; 124:970.79

8. Beyth, R. J., Quinn, L. M., Landefeld, C. S. Prospective evaluation of an index for predicting the risk of major bleeding in outpatients treated with warfarin. Am. J. Med. 1998; 105:91-9.

9. McMahan, D. A., Smith, D. M., Carey, M. A., Zhou, X. H. Risk of major hemorrhage for outpatients treated with warfarin. J. Gen. Intern. Med. 1998; 13:311-6.

10. Poller, I., MacCallum, P. K., Thomson, J. M., Kerns, W. Reduction of factor VII coagulant activity (VIIC), a risk factor for ischemic heart disease, by fixed dose warfarin: a double blind crossover study. Br. Heart J. 1990; 63:231-33.

11. Millenson, M. M., Bauer, K. A., Kistler, J. P., Barzegar, S., Tulin, L., Rosenberg, R. D. Monitoring “mini-intensity” anticoagulation with warfarin: comparison of the prothrombin time using a sensitive thromboplastin with prothrombin fragment F 1+2 levels. Blood 1992; 79:2034-38.

12. Bern, M. M., Lokich, J. J., Wallach, S. R. et al. Very low doses of warfarin can prevent thrombosis in central venous catheters: a randomized prospective trial. Ann. Intern. Med. 1990; 112:423-8.

13. Levine, M., Hirsh, J., Gent, M. et al. Double-blind randomized trial of a very-low-dose warfarin for prevention of thromboembolism in stage IV breast cancer. Lancet 1994; 343:886-9.

14. Ridker, P. M. for the PREVENT Investigators. Long term, low dose warfarin among venous thrombosis patients with and without factor V Leiden mutation:rationale and design for the Prevention of Recurrent Venous Thromboembolism (PREVENT) trial. Vascular Medicine 1998; 3:67-73.

15. Lan, K. K. G., DeMets, D. L. Discrete sequential boundaries for clinical trials. Biometrika, 1983;76:659-663.

16. Eriksson, H., Wahlander, K., Lundstrom, T. et al. for the THRIVE III Investigators. Abstract, American Society of Hematology, December 2002.

17. Bertina, R. M., Koejeman, B. P. C., Koster, T. et al. Mutation in blood coagulation factor V associated with resistance to activated protein C. Nature 1994; 369:64-7.

18. Svensson, P. J., Dahlback, B. Resistance to activated protein C as a basis for venous thrombosis. N. Engl. J. Med. 1994; 330:517-22.

19. Ridker, P. M., Hennekens, C. H., Lindpaintner, K., Stampfer, M. J., Eisenberg, P. R., Miletich, J. P. Mutation in the gene coding for coagulation factor V and the risk of myocardial infarction, stroke, and venous thrombosis in apparently healthy men. N. Engl. J. Med. 1995; 332:912-917

20. Poort, S. R., Rosendaal, F. R., Reitsma, P. H., Bertina, R. M. A common genetic variation in the 3′-untranslated region of the prothrombin gene is associated with elevated plasma prothrombin levels and an increase in venous thrombosis. Blood 1996; 88:3698-703

21. Price, D., Ridker, P. M. Factor V Leiden Mutation and venous thromboembolism: a clinical perspective. Ann. Intern. Med. 1997; 127:895-903.

22. Hillarp, A., Zoller, B., Svensson, P. J., Dahlback, B. The 20210 A allele of the prothrombin gene is a common risk factor among Swedish outpatients with verified deep venous thrombosis. Thromb Haemost 1997; 78:990-992 23.

23. Ridker, P. M., Miletich, J. P., Stampfer, M. J., Goldhaber, S. Z., Lindpaintner, K., Hennekens, C. H. Factor V Leiden and risks of recurrent idiopathic venous thromboembolism. Circulation 1995; 92:2800-2802.

24. Simioni, P., Prandoni, P., Lensing, A. W. A. et al. The risk of recurrent venous thromboembolism in patients with an Arg⁵⁰⁶ Gln mutation in the gene for factor V (factor V Leiden). N. Engl. J. Med. 1997; 336:399-403

25. Miles, J. S., Miletich, J. P., Goldhaber, S. Z., Hennekens, C. H., Ridker P. M. G20210A Mutation in the prothrombin gene and the risk of recurrent venous thromboembolism. JACC 2001; 37:215-8.

26. Eichinger, S., Pabinger, I., Stumpflen, A., Hirschi, M., Bialonczyk, C., Schneider, B., Mannhalter, C., Minar, E., Lechner, K., Kyrle, P. A. The risk of recurrent venous thromboembolism in patients with and without factor V Leiden. Thromb-Haemost 1997; 77:624-8 27.

27. Lindmarker, P., Schulman, S., Sten-Linder, M., Wiman, B., Egberg, N., Johnsson, H. and the DURAC Trial Study Group. The risk of recurrent venous thromboembolism in carriers and non-carriers of the G1691A allele in the coagulation factor V gene and the G20210A allele in the prothrombin gene. Thromb Haemost 1999; 81:684-9

28. De Stefano, V., Martinelli, I., Mannucci, P. M. et al. The risk of recurrent deep venous thrombosis among heterozygous carriers of both factor V Leiden and the G20210A prothrombin mutation. N. Engl. J. Med. 1999; 341:801-6

### APPENDIX A—BLINDED LOW DOSE-WARFARIN TITRATION REGIMEN USED DURING THE BI-MONTHLY FOLLOW-UP VISITS IN THE PREVENT TRIAL

If blinded INR<1.3, increase current dose 2 mg/day and repeat blinded INR. in 1 week.

If blinded INR≦1.3 and <1.5, increase current dose 1 mg/day and repeat INR in 8 weeks.

If blinded INR≧1.5 and ≦2.0, maintain current dose and repeat INR in 8 weeks.

If blinded INR>2.0 and ≦3.0, decrease current dose 1 mg/day and repeat INR in 8 weeks.

If blinded INR>3.0 and ≦4,0, decrease current dose 2 mg/day and repeat INR in 1 week.

If blinded INR>4.0, stop study drug for 3 days and repeat INR. If repeat INR remains >4, discontinue therapy. If repeat INR<4.0, decrease current dose 2 mg/day and repeat INR in 1 week.

What is claimed is:

1. A method for inhibiting, preventing or reducing incidence of recurrent venous thromboembolism, which comprises administering to a patient who has previously undergone standard therapy for venous thromboembolism a low dose of warfarin which is less than the dosage required in standard therapy for venous thromboembolism.

2. The method as defined in claim 1 wherein the low dose of warfarin administered is within the range from about 1.4 to less than about 2.0 INR.

3. The method as defined in claim 1 wherein the low dose of warfarin administered is within the range from about 1.6 to about 1.8 INR.

4. The method as defined in claim 1 wherein the low dose of warfarin administered is about 1.7 INR.

5. The method as defined in claim 1 wherein the low dose of warfarin is administered for at least 2 years and up to the lifetime of the patient.

6. The method as defined in claim 1 wherein the low dose of warfarin administered is within the range from about 0.5 to about 10 mg daily.

7. The method as defined in claim 1 wherein the low dose of warfarin administered is within the range from about 3 to about 6 mg daily.

8. The method as defined in claim 1 wherein the low dose of warfarin administered is about 4 mg daily.
